(19) United States
(12) Patent Application Publication
(10) Pub. No.: US 2002/0128547 A1
(43) Pub. Date: Sep. 12, 2002

(54) Medical image display apparatus and method

(75) Inventors: Yukihito Furuhashi, Hachioji-shi (JP); Hiroshi Matsuzaki, Hachioji-shi (JP); Akito Saito, Hino-shi (JP); Takao Shibasaki, Tokyo (JP)

Correspondence Address: Scully, Scott, Murphy & Presser, 400 Garden City Plaza, Garden City, NY, 11530, US

(73) Assignee: OLYMPUS OPTICAL CO., LTD., TOKYO (JP)

(21) Appl. No.: 10/091,861

(22) Filed: Mar. 6, 2002

(30) Foreign Application Priority Data
Mar. 6, 2001 (JP) 2001-061939

(51) Int. Cl. A61B 5/05

(52) U.S. Cl. 600/407

(57) ABSTRACT

A medical image display apparatus includes a first image output unit which outputs a real image of a subject that is grabbed by an image input unit. A three-dimensional position posture detection unit detects a position and posture of each of the image input unit and the subject and outputs position posture information. A second image output unit generates a data image of the subject based on prerecorded data of the subject using the position posture information and outputs the data image. An image display unit superposes the real image of the subject and the data image of the subject on each other and displays a superposed image. The display area of the data image of the subject output from the second image output unit is larger than that of the real image of the subject output from the first image output unit.

### CROSS-REFERENCE TO RELATED APPLICATIONS

This application is based upon and claims the benefit of priority from the prior Japanese Patent Application No. 2001-061939, filed Mar. 6, 2001, the entire contents of which are incorporated herein by reference.

### BACKGROUND OF THE INVENTION

1. Field of the Invention

The present invention relates to a medical image display apparatus and a medical image display method.

2. Description of the Related Art

Jpn. Pat. Appln. KOKAI Publication No. 10-143652 discloses a structure capable of arbitrarily setting a superposed area of a three-dimensional medical image of a data image when the three-dimensional medical image is superposed on a two-dimensional live image of a real image displayed on the entire screen. Further, an endoscope is known which virtually observes a lumen by forming and displaying a data image within the entire field of view.

However, the above Publication simply teaches superposing and displaying the data image corresponding to part of the real image. In an endoscope having a small diameter, the field of view for a real image is narrow and thus it is hard to know which part of a subject to be examined is currently observed. In an endoscope for forming and displaying a data image within the entire field of view, it is impossible to know a relationship in position between the real image of a subject to be examined and the data image.

### BRIEF SUMMARY OF THE INVENTION

Accordingly, an object of the present invention is to provide a medical image display apparatus and a medical image display method in which an operator can easily know a relationship in position between a real image and a data image and which part of a subject corresponds to an observed area by superposing a small real image on a larger data image.

In order to attain the above object, a medical image display apparatus according to a first aspect of the present invention comprises:

a first image output unit which outputs a real image of a subject that is grabbed by an image input unit;

a three-dimensional position posture detection unit which detects a position and posture of each of the image input unit and the subject and outputs position posture information;

a second image output unit which generates a data image of the subject based on prerecorded data of the subject using the position posture information output from the three-dimensional position posture detection unit, and outputs the data image; and

an image display unit connected to the first image output unit and the second image output unit, which superposes the real image of the subject output from the first image output unit and the data image of the subject output from the second image output unit on each other and displays a superposed image,

wherein a display area of the data image of the subject output from the second image output unit is larger than a display area of the real image of the subject output from the first image output unit.

A medical image display apparatus according to a second aspect of the present invention depends from the medical image display apparatus according to the first aspect of the present invention, in which the image display unit has a first display area and a second display area formed around the first display area, and the real image of the subject output from the first image output unit is displayed in the first display area and the data image of the subject output from the second image output unit is displayed in the second display area.

A medical image display apparatus according to a third aspect of the present invention depends from the second aspect of the present invention, in which the first display area and the second display area include an overlapped portion in a boundary therebetween.

A medical image display apparatus according to a fourth aspect of the present invention depends from the first aspect of the present invention, in which the image display unit has a first display area and a second display area formed around the first display area, and the real image of the subject output from the first image output unit is displayed in the first display area, only an outline of the data image of the subject output from the second image output unit is displayed in the first display area, and a whole of the data image of the subject is displayed in the second display area.

A medical image display apparatus according to a fifth aspect of the present invention depends from the medical image display apparatus according to the first aspect of the present invention, in which the data image of the subject output from the second image output unit is a wire frame image.

A medical image display apparatus according to a sixth aspect of the present invention depends from the medical image display apparatus according to the first aspect of the present invention, in which the data image of the subject output from the second image output unit is an image formed by dots.

A medical image display apparatus according to a seventh aspect of the present invention depends from the medical image display apparatus according to the first aspect of the present invention, in which the data image of the subject output from the second image output unit is a surface image that is displayed through the real image of the subject output from the first image output unit.

A medical image display method according to an eighth aspect of the present invention comprises:

a step of inputting a real image of a subject from an image input unit;

a step of detecting a three-dimensional position and posture of each of the image input unit and the subject;

a step of generating a data image of the subject based on prerecorded data of the subject using the detected three-dimensional position and posture of each of the image input unit and the subject; and

a step of superposing the real image of the subject and the data image of the subject and displaying a superposed image,

wherein a display area of the data image of the subject is larger than a display area of the real image of the subject.

A medical image display apparatus according to a ninth aspect of the present invention depends from the medical image display apparatus according to the eighth aspect of the present invention, in which the real image of the subject is displayed in a first display area formed in substantially a central part of an image display area and the data image of the subject is displayed in a second display area formed around the first display area.

A medical image display apparatus according to a tenth aspect of the present invention depends from the medical image display apparatus according to the ninth aspect of the present invention, in which the first display area and the second display area include an overlapped portion in a boundary therebetween.

A medical image display apparatus according to an eleventh aspect of the present invention depends from the medical image display apparatus according to the eighth aspect of the present invention, in which the real image of the subject is displayed in a first display area formed in substantially a central part of an image display area, only an outline of the data image of the subject is displayed in the first display area, and a whole of the data image of the subject is displayed in a second display area formed around the first display area.

A medical image display apparatus according to a twelfth aspect of the present invention depends from the medical image display apparatus according to the eighth aspect of the present invention, in which the data image of the subject is a wire frame image.

A medical image display apparatus according to a thirteenth aspect of the present invention depends from the medical image display apparatus according to the eighth aspect of the present invention, in which the data image of the subject is an image formed by dots.

A medical image display apparatus according to a fourteenth aspect of the present invention depends from the medical image display apparatus according to the eighth aspect of the present invention, in which the data image of the subject is a surface image that is displayed through the real image.

### BRIEF DESCRIPTION OF THE SEVERAL VIEWS OF THE DRAWING

FIG. 1 is a block diagram showing a configuration of a medical image display apparatus according to an embodiment of the present invention.

FIG. 2 is an illustration of a real image of a subject to be examined and a data image outside a field of view, which are superposed and displayed on a display screen of a CRT.

FIG. 3 is an illustration of a first modification to the superposition and display of the real image of the subject and the data image outside the field of view.

FIG. 4 is an illustration of a second modification to the superposition and display of the real image of the subject and the data image outside the field of view.

FIG. 5 is an illustration of a third modification to the superposition and display of the real image of the subject and the data image outside the field of view.

FIG. 6 is an illustration of a modification to the display of the real image.

### DETAILED DESCRIPTION OF THE INVENTION

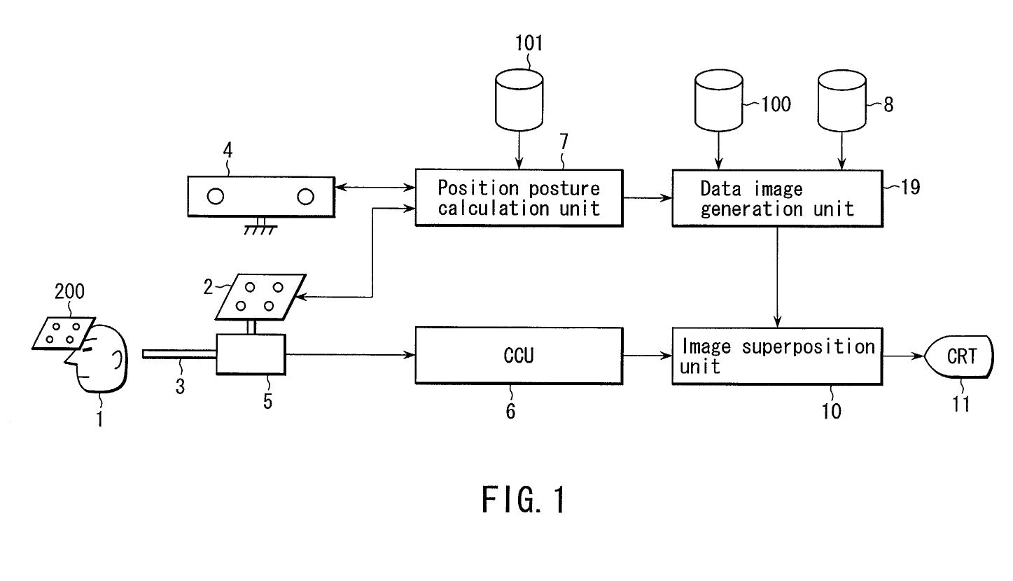

An embodiment of the present invention will now be described in detail with reference to the accompanying drawings. FIG. 1 is a block diagram showing a configuration of a medical image display apparatus according to the embodiment of the present invention.

A camera head 5 converts an optical image of a subject 1 to be examined, which is captured by a rigid endoscope 3 serving as an image input unit, into an electrical signal. A CCU (camera control unit) 6 serving as a first image output unit converts the electrical signal into an NTSC video signal, a PAL video signal, and the like and supplies the video signal to an image superposition unit 10 as a real image corresponding to the field of view of the rigid endoscope 3.

An infrared LED 200 is fixedly attached to the subject 1. An LED attachment position posture information storage unit 101 stores three-dimensional position posture information concerning the infrared LED 200 and three-dimensional data of the subject 1, which is measured in advance.

An infrared LED 2 is attached to the camera head 5. A position sensor 4 is provided so as to cause the infrared LEDs 2 and 200 to fall within a measurement range. A position posture calculation unit 7 performs an operation using infrared information sensed by the position sensor 4 from the infrared LEDs 2 and 200 and the position posture information stored in the LED attachment position posture information storage unit 101. It is thus possible to three-dimensionally detect a position and posture of the moving rigid endoscope 3 relative to the subject.

The position posture information obtained from the position posture calculation unit 7 is input to a data image generation unit 19 serving as a second image output unit. A subject three-dimensional data storage unit 8 stores subject three-dimensional data that is created based on slice images of the subject 1, which are captured in advance by CT, MRI or the like. A rigid endoscope optical parameter storage unit 100 stores optical parameters (central position, magnification, distortion, etc.) of the rigid endoscope 3, which are measured in advance.

The data image generation unit 19 reads subject three-dimensional data from the subject three-dimensional data storage unit 8 based on both the position posture information of the position posture calculation section 7 and the information of the rigid endoscope optical parameter storage unit 100 to generate a data image outside a field of view of a real image of the rigid endoscope 3. The unit 19 then supplies the data image to the image superposition unit 10. Since the data image generated by the data image generation unit 19 is based on the information of the rigid endoscope optical parameter storage unit 100, it coincides with an image of an enlarged field of view of the rigid endoscope 3.

The image superposition unit 10 superposes a real image within the field of view acquired from the CCU 6 and a data image outside the field of view acquired from the data image generation unit 19 and sends the superposed image to a CRT 11.

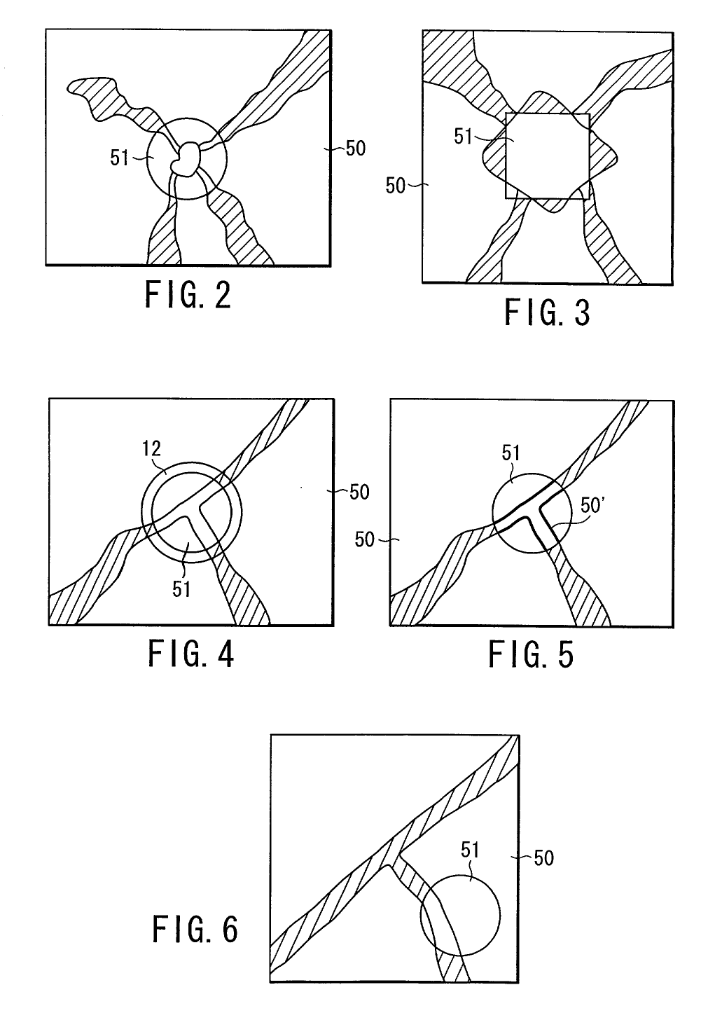

FIG. 2 is an illustration of a real image of the subject 1 and a data image outside the field of view, which are superposed and displayed on a display screen of the CRT 11. A real image 51 is displayed in a circular area that is formed in substantially the central part of the display screen as a first display area, and a data image 50 outside the field of view is displayed in a second display area around the first display area such that the data image is connected to the real image. By superposing the small real image 51 on the large data image 50, it is possible to easily know a relationship in position between these images 50 and 51 and which part of the subject corresponds to an observed area.

FIG. 3 is an illustration of a first modification to the superposition and display of the real image 51 of the subject 1 and the data image 50 outside the field of view. In this modification, the display area of the real image 51 is shaped like a rectangle.

FIG. 4 is an illustration of a second modification to the superposition and display of the real image 51 of the subject 1 and the data image 50 outside the field of view. In this modification, a donut-shaped overlapped section 12 is provided in a boundary between the real image 51 displayed in substantially the central part of the display screen as a first area and the data image 50 displayed in a second area formed around the first display area. Since the real image 51 and data image 50 are superposed and displayed in this section 12, a relationship in position between the real and data images 51 and 50 can easily be understood. In other words, an operator can visually confirm whether the data image 50 coincides with the real image 51 in the overlapped section 12.

FIG. 5 is an illustration of a third modification to the superposition and display of the real image of the subject and the data image outside the field of view. In this modification, the real image 51 is displayed in substantially the central part of the display screen as a first area, the data image 50 is displayed in a second area formed around the first display area, and an outline section 50′ corresponding to the data image 50 is superposed on the real image 51 displayed in the first display area. This superposition allows an operator to easily know a relationship in position between an internal image of the real image 51 and the data image 50 without hindering an operator from being easily seeing the real image 51. In other words, an operator can visually confirm whether the data image 50 coincides with the internal image of the real image 51.

In the foregoing embodiment, the real image 51 is displayed in substantially the central part of the display screen. The present invention is not limited to this display. For example, as shown in FIG. 6, the real image 51 can be displayed in the lower right part of the display screen.

As the data image displayed in the second display area, a wire frame image, an image of a subject formed by dots, or a surface image that is displayed through the real image displayed in the first display area can be used.

According to the present invention, since a small real image is superposed on a larger data image, an operator can easily know a relationship in position between the real and data images and which part of a subject corresponds to an observed area.

According to the present invention, since the data image is displayed around the real image, it is possible to widen the field of view of a small real image and thus easily know which part of a subject corresponds to an observed area.

According to the present invention, since the real and data images overlap each other, it is easy for an operator to know a relationship in position between them.

According to the present invention, since only the outline of the data image is superposed on the real image, an operator can easily know a relationship in position between the real image and the internal image of the real image without hindering the operator from being easily seeing the real image.

What is claimed is:

1. A medical image display apparatus comprising:
a first image output unit which outputs a real image of a subject that is grabbed by an image input unit;
a three-dimensional position posture detection unit which detects a position and posture of each of the image input unit and the subject and outputs position posture information;
a second image output unit which generates a data image of the subject based on prerecorded data of the subject using the position posture information output from the three-dimensional position posture detection unit, and outputs the data image; and
an image display unit connected to the first image output unit and the second image output unit, which superposes the real image of the subject output from the first image output unit and the data image of the subject output from the second image output unit on each other and displays a superposed image,
wherein a display area of the data image of the subject output from the second image output unit is larger than a display area of the real image of the subject output from the first image output unit.

2. The medical image display apparatus according to claim 1, wherein the image display unit has a first display area and a second display area formed around the first display area, and the real image of the subject output from the first image output unit is displayed in the first display area and the data image of the subject output from the second image output unit is displayed in the second display area.

3. The medical image display apparatus according to claim 2, wherein the first display area and the second display area include an overlapped portion in a boundary therebetween.

4. The medical image display apparatus according to claim 1, wherein the image display unit has a first display area and a second display area formed around the first display area, and the real image of the subject output from the first image output unit is displayed in the first display area, only an outline of the data image of the subject output from the second image output unit is displayed in the first display area, and a whole of the data image of the subject is displayed in the second display area.

5. The medical image display apparatus according to claim 1, wherein the data image of the subject output from the second image output unit is a wire frame image.

6. The medical image display apparatus according to claim 1, wherein the data image of the subject output from the second image output unit is an image formed by dots.

7. The medical image display apparatus according to claim 1, wherein the data image of the subject output from the second image output unit is a surface image that is displayed through the real image of the subject output from the first image output unit.

8. A medical image display method comprising:
a step of inputting a real image of a subject from an image input unit;
a step of detecting a three-dimensional position and posture of each of the image input unit and the subject;
a step of generating a data image of the subject based on prerecorded data of the subject using the detected three-dimensional position and posture of each of the image input unit and the subject; and
a step of superposing the real image of the subject and the data image of the subject and displaying a superposed image,
wherein a display area of the data image of the subject is larger than a display area of the real image of the subject.

9. The medical image display method according to claim 8, wherein the real image of the subject is displayed in a first display area formed in substantially a central part of an image display area and the data image of the subject is displayed in a second display area formed around the first display area.

10. The medical image display method according to claim 9, wherein the first display area and the second display area include an overlapped portion in a boundary therebetween.

11. The medical image display method according to claim 8, wherein the real image of the subject is displayed in a first display area formed in substantially a central part of an image display area, only an outline of the data image of the subject is displayed in the first display area, and a whole of the data image of the subject is displayed in a second display area formed around the first display area.

12. The medical image display method according to claim 8, wherein the data image of the subject is a wire frame image.

13. The medical image display method according to claim 8, wherein the data image of the subject is an image formed by dots.

14. The medical image display method according to claim 8, wherein the data image of the subject is a surface image that is displayed through the real image.
